(19) United States
(12) Patent Application Publication
(10) Pub. No.: US 2019/0351130 A1
(43) Pub. Date: Nov. 21, 2019

(54) THOROUGH-DRIPPING MEDICAL INFUSION APPARATUS WITH INFUSION BOTTLE HAVING INBUILT BALLOON

(71) Applicant: Jiangping TANG, Dalian (CN)

(72) Inventor: Jiangping TANG, Dalian (CN)

(21) Appl. No.: 16/421,582

(22) Filed: May 24, 2019

Related U.S. Application Data
(63) Continuation of application No. PCT/CN2017/000218, filed on Mar. 7, 2017.

(30) Foreign Application Priority Data
Nov. 25, 2016 (CN) PCT/CN2016/000659

(51) Int. Cl. A61M 5/14

(52) U.S. Cl. CPC A61M 5/1411; A61J 1/10; A61M 5/1414

(57) ABSTRACT

A medical infusion apparatus with infusion bottle having inbuilt balloon is disclosed, wherein a polyvinyl chloride plastic capsule or spherical balloon is built in the production of infusion bottle, glass bottle, polypropylene (PP) plastic bottle, polyvinyl chloride (PVC) plastic capsule, non-polyvinyl-chloride polyolefin infusion soft bag, and the PVC plastic capsule or spherical balloon is elastic, uniformly distributed with 6 pinholes on a surface thereof, has a volume of 10% of a liquid capacity of the infusion bottle or infusion bag, and is disposed in the infusion bottle or infusion bag in a fixed state or an unfixed natural state; the infusion bottle or infusion bag is injected with a base liquid during production, and also the plastic capsule or spherical balloon is naturally filled with the base liquid.

### CROSS-REFERENCE TO RELATED APPLICATIONS

This application is a continuation of International Application No. PCT/CN2017/000218, filed on Mar. 7, 2017, which claims priority to International Application No. PCT/CN2016/000659 filed on Nov. 25, 2016, the disclosure of which is hereby incorporated by reference in its entirety.

### TECHNICAL FIELD

The present disclosure relates to a new medical drug delivery system, in particular to a thorough-dripping medical infusion apparatus with infusion bottle having inbuilt balloon capable of eliminating the residual liquid of the infusion system at the end of the medical infusion process.

### BACKGROUND

At present, a well-known medical disposable infusion apparatus is a common disposable consumable mainly used for intravenous infusion, for establishing a passage between a vein and a liquid medicine after sterilization. Generally, it consists of a venous needle, a protective cap, an infusion hose, a liquid medicine filter, a flow rate regulator, a drip pot, a bottle stopper puncture device, an air inlet air filter, and an infusion bottle or infusion bag connection structure. Medical infusion mainly uses the principle of gravity (earth gravity) as well as auxiliary pressure (atmospheric pressure) to cause the infusion liquid to fall freely from a height, thereby completing the process of infusion for the human body. Due to the complicated and costly pharmaceutical process of infusion drugs directly used for human administration, the structure and design of traditional infusion apparatus have long been insufficient, resulting in the inability to completely give the medicine to the human body in the later stage of the infusion process. The liquid medicine resided in the infusion system is directly discarded, which is a long-standing phenomenon in the medical system. It directly brings problems of: 1. lack of medicine of the human body; 2. waste of medical resources and funds; 3. intangible burden to pharmaceutical companies, medical enterprises and the people, and huge amount of accumulated waste; this problem has long been ignored.

### SUMMARY

In order to eliminate the residual problem of the liquid medicine in the infusion system at the end of the infusion process, the present disclosure provides a thorough-dripping medical infusion apparatus with infusion bottle having inbuilt balloon, which conforms to the traditional infusion protocol and can drip out the liquid medicine completely.

The technical solution adopted by the patent of the present disclosure to solve its technical problems is:

1. A PVC plastic capsule or spherical balloon (shaped or round) is built in infusion bottle, glass bottle, polypropylene (PP) plastic bottle, polyvinyl chloride (PVC) plastic capsule, non-polyvinyl-chloride polyolefin infusion soft bag, and the PVC plastic capsule or spherical balloon is elastic, uniformly distributed with 6 pinholes on a surface thereof, has a volume of 10% of a liquid capacity of the infusion bottle or infusion bag, and is disposed in the infusion bottle or infusion bag in a fixed state or an unfixed natural state; the infusion bottle or infusion bag is injected with a base liquid (physiological saline/glucose injection) during production, and also the PVC plastic capsule or spherical balloon is naturally filled with the base liquid; and during medical use, an injection operation of medicine into the infusion bottle or infusion bag and an infusion operation of medicine into a human body are the same as for traditional medical procedures.

2. At this time, since the mixed liquid medicine in the infusion bottle or infusion bag and the base fluid in the PVC plastic capsule or spherical balloon are in a positive pressure equilibrium state, the liquid medicine no longer enters the PVC plastic capsule or spherical balloon.

3. Before an end of dripping of infusion flow, at this time, the infusion bottle or infusion bag is to be emptied, the base liquid in the PVC plastic capsule or spherical balloon is seeped from the reserved pinholes and dripped into the infusion bottle or infusion bag and an infusion hose under an action of negative pressure, and an seeped amount of the base liquid is suitable for the infusion flow amount, in a dripping process under an action of continuous drip, the liquid medicine in the infusion system and the infusion hose is completely forced to drip out by the base liquid, and finally a content that remains in the infusion hose is subsequent base liquid (physiological saline/glucose injection) from the PVC plastic capsule or spherical balloon.

4. The medical infusion apparatus with infusion bottle having inbuilt balloon is realized so that by an infusion process in which the liquid medicine is firstly and the base liquid is subsequently delivered, the liquid medicine is able to be completely infused into the human body.

The present disclosure using thorough-dripping medical infusion apparatus having balloon built in infusion bottle has the advantages of conveniently and effectively delivering the liquid medicine to the human body completely while complying with the traditional infusion operation procedure and program, and having a simple structure.

### BRIEF DESCRIPTION OF THE DRAWINGS

FIG. 1 is a longitudinal sectional view of the infusion apparatus of the present disclosure.

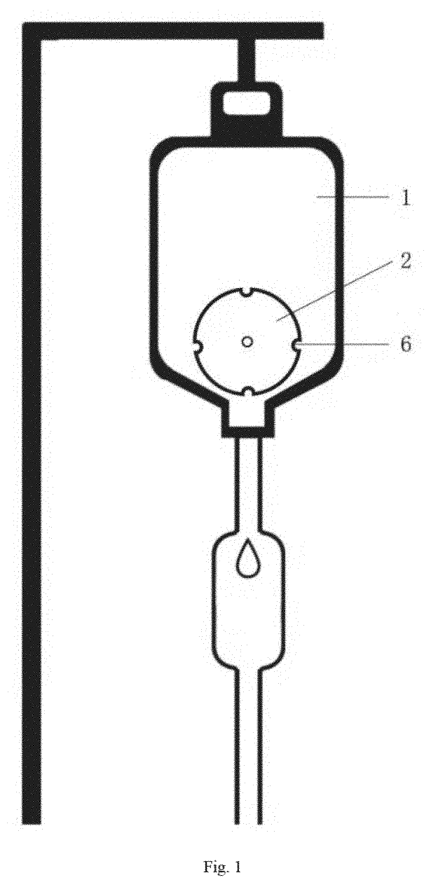

FIG. 2 shows different views during use of the infusion apparatus of the present disclosure.

### REFERENCE NUMERALS

1: infusion apparatus system with infusion bottle or infusion bag;
2: PVC plastic capsule or spherical balloon (shaped or round) built in an infusion bottle or infusion bag; the PVC plastic capsule or spherical balloon is elastic, uniformly distributed with 6 pinholes on a surface thereof, has a volume of 10% of a liquid capacity of the infusion bottle or infusion bag, and is disposed in the infusion bottle or infusion bag in a fixed state or an unfixed natural state;
6: 6 pinholes on the composite capsule or balloon.

### DETAILED DESCRIPTION OF EMBODIMENTS

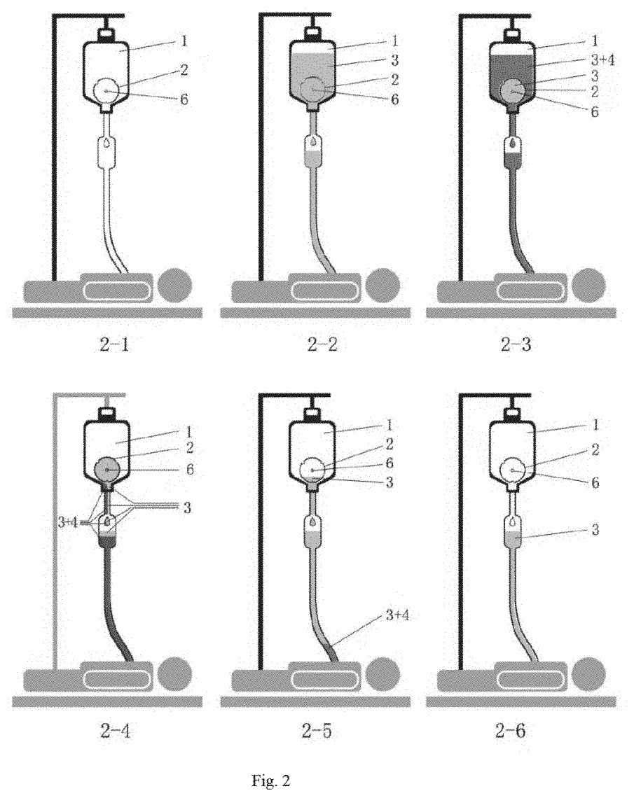

An embodiment according to the present disclosure is shown in FIG. 2.

2-1

1: infusion apparatus system with infusion bottle or infusion bag without injection of any liquid:

2: PVC plastic capsule or spherical balloon (shaped or round) built in an infusion bottle or infusion bag; the composite plastic capsule or spherical balloon is elastic, uniformly distributed with 6 pinholes on a surface thereof, has a volume of 10% of a liquid capacity of the infusion bottle or infusion bag, and is disposed in the infusion bottle or infusion bag in a fixed state or an unfixed natural state.

2-2

1: the infusion apparatus system with infusion bottle or infusion bag injected with base liquid 3 (physiological saline or glucose injection)

2: composite capsule or spherical balloon;

3: base liquid (physiological saline or glucose injection).

2-3

During medical use, medicine 4 is injected into the infusion apparatus system with infusion bottle or infusion bag containing the base fluid 3 (physiological saline or glucose injection), so that a mixed liquid medicine 3+4 is formed. At this time, the mixed liquid medicine 3+4 in the infusion bottle or infusion bag and the base fluid 3 (physiological saline or glucose injection) in the composite plastic capsule or spherical balloon are in a positive pressure equilibrium state, and the mixed liquid medicine no longer enters the composite plastic capsule or spherical balloon.

2-4

During medical use, at a later stage of delivery of the mixed liquid medicine 3+4 in the infusion apparatus system with infusion bottle or infusion bag 1, a negative pressure will be generated in the infusion bottle or infusion bag that is to be empitied, at this time, the base fluid 3 (physiological saline or glucose injection) in the composite plastic capsule or spherical balloon 2 is seeped from the reserved pinholes 6 and dripped into the infusion apparatus system with infusion bottle or infusion bag under an action of negative pressure, at this time, the liquid medicine is firstly and the base liquid is subsequently delivered and the dripping and delivery is continued.

2-5

Before the end of medical use, the former mixed liquid medicine 3+4 is forced by the subsequent base liquid 3 (physiological saline or glucose injection) to drip out completely and delivered to the human body;

2-6

At the end of medical use, a content that remains in the infusion apparatus system 1 is subsequent base liquid 3 (physiological saline/glucose injection) from the composite capsule or spherical balloon 2.

What is claimed is:

1. A thorough-dripping medical infusion apparatus with infusion bottle having inbuilt balloon, wherein a PVC plastic capsule or spherical balloon is built in a conventional infusion bottle or infusion bag, and the PVC plastic capsule or spherical balloon is elastic, uniformly distributed with 6 pinholes on a surface thereof has a volume of 10% of a liquid capacity of the infusion bottle or infusion bag, and is disposed in the infusion bottle or infusion bag in a fixed state or an unfixed natural state; the infusion bottle or infusion bag is injected with a base liquid during production, and also the PVC plastic capsule or spherical balloon is naturally filled with the base liquid; and during medical use, an injection operation of medicine into the infusion bottle or infusion bag and an infusion operation of medicine into a human body are the same as for traditional medical procedures.

2. The thorough-dripping medical infusion apparatus with infusion bottle having inbuilt balloon according to claim 1, wherein after the injection operation, the mixed liquid medicine in the infusion bottle or infusion bag and the base fluid in the PVC plastic capsule or spherical balloon are in a positive pressure equilibrium state, and the mixed liquid medicine no longer enters the PVC plastic capsule or spherical balloon.

3. The thorough-dripping medical infusion apparatus with infusion bottle having inbuilt balloon according to claim 1, wherein before an end of dripping of infusion flow, at this time, the infusion bottle or infusion bag is to be emptied, the base liquid in the PVC plastic capsule or spherical balloon is seeped from the reserved pinholes and dripped into the infusion bottle or infusion bag and an infusion hose under an action of negative pressure, and an seeped amount of the base liquid is suitable for the infusion flow amount, in a dripping process under an action of continuous drip, the liquid medicine in the infusion system and the infusion hose is completely forced to drip out by the base liquid, and finally a content that remains in the infusion hose is subsequent base liquid from the PVC plastic capsule or spherical balloon.

4. The thorough-dripping medical infusion apparatus with infusion bottle having inbuilt balloon according to claim 1, wherein the infusion bottle or infusion bag is provided with the built-in PVC plastic capsule or spherical balloon, that is, the medical infusion apparatus with infusion bottle having inbuilt balloon is realized so that by a infusion process in which the liquid medicine is firstly and the base liquid is subsequently delivered, the liquid medicine is able to be completely infused into the human body.

5. The thorough-dripping medical infusion apparatus with infusion bottle having inbuilt balloon according to claim 1, wherein the infusion bottle is a glass bottle or a polypropylene plastic bottle.

6. The thorough-dripping medical infusion apparatus with infusion bottle having inbuilt balloon according to claim 1, wherein the infusion bag is a polyvinyl chloride plastic bag or a polyolefin non-PVC soft bag.

7. The thorough-dripping medical infusion apparatus with infusion bottle having inbuilt balloon according to claim 1, wherein the base liquid is physiological saline or glucose injection.

8. The thorough-dripping medical infusion apparatus with infusion bottle having inbuilt balloon according to claim 1, wherein the PVC plastic capsule or spherical balloon is shaped or rounded.
